(19) United States
(12) Patent Application Publication
(10) Pub. No.: US 2024/0208601 A1
(43) Pub. Date: Jun. 27, 2024

(54) FOLDING JOINT

(71) Applicant: SHENZHEN MEIDAHANG TECHNOLOGY CO., LTD., Bao'an District Shenzhen, Guangdong (CN)

(72) Inventors: Yiping MO, Shenzhen (CN); David Tak Wei HON, Shenzhen (CN); Guoping LIU, Shenzhen (CN)

(21) Appl. No.: 18/262,115

(22) PCT Filed: Jan. 14, 2022
(86) PCT No.: PCT/CN2022/071972

§ 371 Date: Jul. 19, 2023

(30) Foreign Application Priority Data
Jan. 19, 2021 (CN) 202120138265.3

(51) Int. Cl. B62K 15/00

(52) U.S. Cl. CPC B62K 15/006

(57) ABSTRACT

A folding joint, the folding joint having two connecting plates, which are respectively connected to a rod member and are hinged on one side, wherein folding surfaces of the two connecting plates each have two or more bosses; each of the bosses of one of the connecting plates is provided with a clamping block, which is driven by a screw rod and has a groove at one side; concave edges of the groove on one side of the clamping block can simultaneously come into contact with flanges of the bosses of each of the connecting plates when the two connecting plates are folded close to each other; and the connecting plates folded close to each other are clamped and fastened.

### CROSS-REFERENCE TO RELATED APPLICATIONS

This application is an U.S. national phase application under 35 U.S.C. § 371 based upon international patent application No. PCT/CN2022/071972, filed on Jan. 14, 2022, which itself claims priority to Chinese patent application No. 2021201382653 filed on Jan. 19, 2021. The contents of the above identified applications are hereby incorporated herein in their entireties by reference.

### TECHNICAL FIELD

The present disclosure relates to the field of folding technology, in particular to a folding joint.

### BACKGROUND

As a means of transportation, bicycle plays an important role in people's daily life. For the convenience of portability, folding bicycles appear in people's lives. And in order to realize the switching between a folding state and a non-folding state of the folding bicycle, a folding joint is needed.

Existing folding joints generally include two connecting rods and two joints connecting two sections of vehicle pipes respectively. An end of each of the two joints is hinged, and a locking device is arranged to externally or internally secure two connecting plates that are close together, so that they cannot be opened. Since the joints are used on the frame, they are subjected to a lot of force during riding bumps. The folding joints must not only ensure sufficient rigidity when they are closed and fastened, but also make the opening and closing operations of the joints quick and convenient. Therefore, the locking device has to be designed thicker and has a complicated structure, and the manufacturing cost is high.

### SUMMARY

The object of the present disclosure is to provide a folding joint configured to double fasten two closed connecting plates, so that the locking is reliable, the structure is simple, and the manufacturing cost is low.

The folding joint provided in the present disclosure includes two connecting plates 2 connected to two sections of rods 1 respectively. A side of the two connecting plates 2 are hinged to each other. At least two corresponding edges of opposite cooperating surfaces of the two connecting plates are provided with boss, and each boss of one of the connecting plates is provided with a movable clamping block with a groove on a side thereof. A concave edge of the groove on the side of the clamping block is capable of being in contact with a flange of each boss of the connecting plate simultaneously when the two connecting plates are close together. The clamping block is respectively driven by left and right threads on a screw rod, so that the concave edge of the groove of the clamping block is in contact with or moves away from the flange of the boss of the connecting plate.

According to the folding joint provided in the present disclosure, the two connecting plates have more than two bosses, when the two connecting plates are closed, the concave edge of the clamping block can be in contact with the flange of the connecting plate boss simultaneously, that is, the two closed connecting plates are clamped so that they cannot be opened. The double locking folding joint uses the left and right threads of the screw rod to drive the movement of the clamping block. The locking or unlocking of the two connecting plates can be achieved by using only the thread of the screw rod to drive a short-distance linear movement of the clamping block. The structure of using the screw rod to control the short-distance movement of the clamping block is very simple, so that the opening and closing operation of the entire folding joint is quick and convenient.

### BRIEF DESCRIPTION OF THE DRAWINGS

FIG. 1 is a perspective view of a folding joint in a half-opened state.

FIG. 2 is a profile view of the folding joint in a closing state.

FIG. 3 is a cross-sectional view of the folding joint.

FIG. 4 is a perspective view of a folding joint in a half-opened state.

FIG. 5 is a perspective view of a biaxial hinged folding joint in a half-opened state.

### DETAILED DESCRIPTION OF THE EMBODIMENTS

In order to make the above objects, features and advantages of the present disclosure clear and easier to understand, the specific embodiments of the present disclosure are described in detail below in combination with the accompanying drawings. Many specific details are set forth in the following description to facilitate a full understanding of the present disclosure. However, the present disclosure can be implemented in many ways different from those described herein, and those skilled in the art can make similar improvements without departing from the connotation of the present disclosure. Therefore, the present disclosure is not limited by the specific embodiments disclosed below.

In the description of the present disclosure, it should be understood that the terms “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, “circumferential direction” are based on the azimuth or position relationship shown in the attached drawings, which are only for the convenience of describing the present disclosure and simplifying the description, rather than indicating or implying that the device or element must have a specific azimuth, be constructed and operated in a specific azimuth, so such terms cannot be understood as a limitation of the present disclosure.

In addition, the terms “first” and “second” are only used for descriptive purposes and cannot be understood as indicating or implying relative importance or implicitly indicating the number of indicated technical features. Thus, the features defined with “first” and “second” may explicitly or implicitly include at least one of the features. In the description of the present disclosure, “multiple” means at least two, such as two, three, etc., unless otherwise expressly and specifically defined.

In the present disclosure, unless otherwise expressly specified and limited, the terms “mount”, “connect”, “contact”, “fix” and other terms should be understood in a broad sense, for example, they can be fixed connections, removable connections, or integrated. They can be mechanical connection or electrical connection. They can be directly connected or indirectly connected through an intermediate medium. They can be the connection within two elements or the interaction relationship between two elements, unless otherwise expressly limited. For those skilled in the art, the specific meaning of the above terms in the present disclosure can be understood according to the specific situation.

In the present disclosure, unless otherwise expressly specified and limited, the first feature “above” or “below” the second feature may be in direct contact with the first and second features, or the first and second features may be in indirect contact through an intermediate medium. Moreover, the first feature is “above” the second feature, but the first feature is directly above or diagonally above the second feature, or it only means that the horizontal height of the first feature is higher than the second feature. The first feature is “below” of the second feature, which can mean that the first feature is directly below or obliquely below the second feature, or simply that the horizontal height of the first feature is less than that of the second feature.

It should be noted that when an element is called “fixed to” or “provided on” another element, it can be directly on another element or there can be a centered element. When an element is considered to be “connected” to another element, it can be directly connected to another element or there may be intermediate elements at the same time. The terms “vertical”, “horizontal”, “up”, “down”, “left”, “right” and similar expressions used herein are for the purpose of illustration only and do not represent the only embodiment.

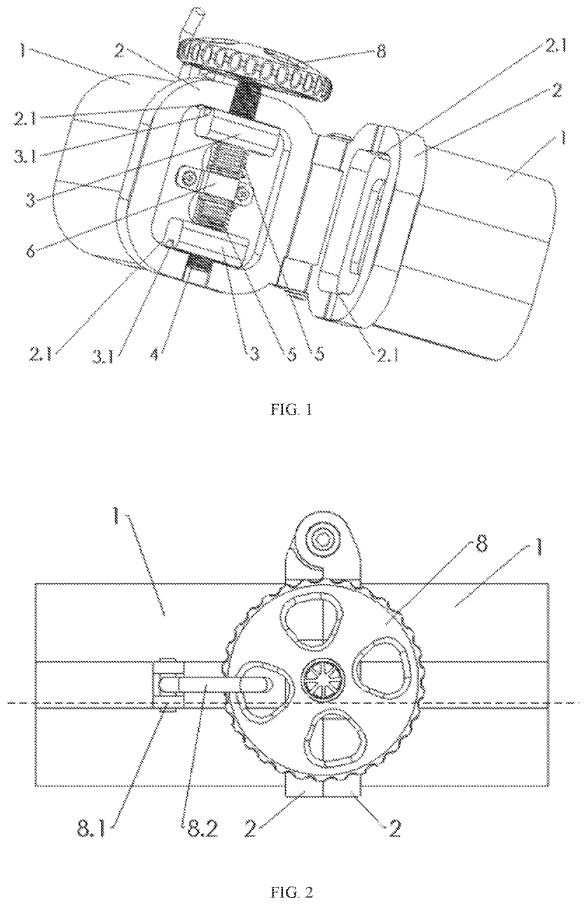

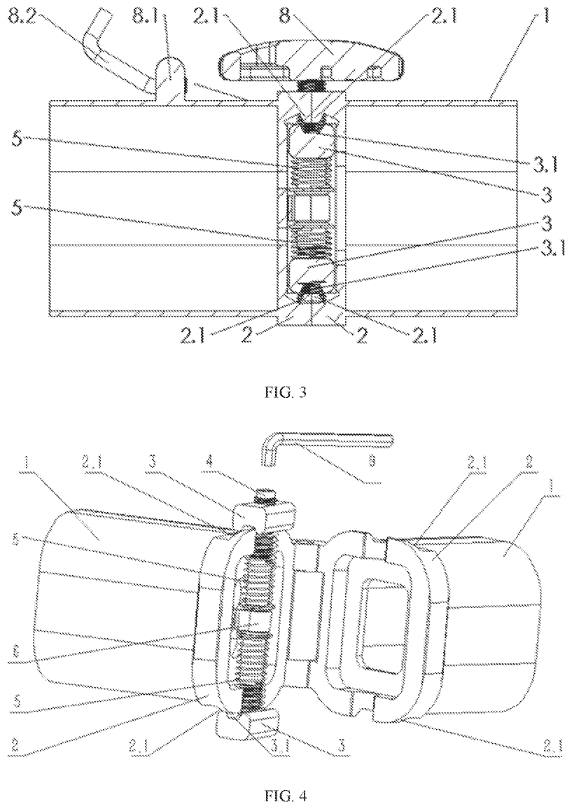

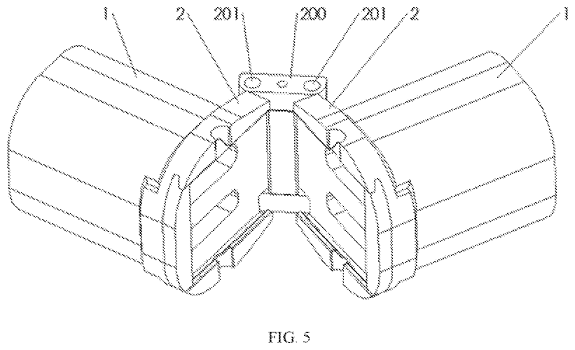

Embodiment 1 is shown in FIGS. 1 to 3.

A folding joint includes two connecting plates 2 connected to two sections of rods 1, respectively. Sides of the two connecting plates 2 are hinged to each other, and opposite cooperating surfaces of the two connecting plates 2 are provided with an upper trapezoid boss 2.1 and a lower trapezoid boss 2.1 inside the two connecting plates 2. Movable clamping blocks 3 are arranged at the upper and lower bosses of one connecting plate 2, and a side of the clamping block 3 adjacent to the trapezoidal boss 2.1 is provided with a trapezoidal groove. An upper section of a screw rod 4 has a right-handed thread connected to a right-hand thread of the lower clamping block 3, and a lower section of the screw rod 4 has a left-handed thread connected to a left-hand thread of the lower clamping block 3. An upper section of screw rod 4 is fixedly connected to a handle, and the screw rod 4 is fixedly connected to the connecting plate 2 through a screw rod buckle 6.

According to the folding joint, the boss of connecting plate 2 can be arranged inside or outside the connecting plate. The two connecting plates 2 are hinged through a biaxial connecting block 200 and two hinge shafts 201. A flange of the boss 2.1 of the connecting plate 2 is trapezoidal or arc-shaped. The rod 1 is provided with a safety buckle seat 8.1 and a safety buckle 8.2.

When the handle 8 is rotated rightwards, the right-handed thread of the screw rod 4 drives the upper clamping block 3 to move upward, so that a concave edge of the trapezoidal groove 3.1 of the upper clamping block 3 is in contact with the flanges of the upper trapezoidal boss 2.1 of the two connecting blocks, respectively, and the lower clamping block 3 is driven to move downward, so that the concave edge of the trapezoidal groove 3.1 of the lower clamping block 3 is in contact with the flanges of the lower trapezoidal boss 2.1 of the two connecting blocks, respectively, which is in a locked state. The two clamping blocks are used to lock the two connecting plates up and down, which is very firm. The clamping block 3 adopts the groove 3.1 to fasten the boss 2.1 of the connecting plate 2, which has a compact structure. The movement of the clamping block 3 is driven by the most common screw rod 4, which has a very simple and reliable structure, so that the connecting plate 2 can become more compact and have good appearance, and the manufacturing process is simple and the cost is low. The safety buckle 8.2 is rotated to insert into a hole of handle 8, so that the handle 8 is locked and cannot rotate, which can prevent the screw rod 4 from rotating to the left accidentally and causing the clamping block 3 to move, which plays a safe role. When the safety buckle 8.2 is rotated out of the hole of the handle 8, and the handle 8 is rotated leftwards, the right-handed thread of the screw rod 4 drives the upper clamping block 3 to move down, so that the concave edge of the trapezoidal groove 3.1 of the upper clamping block 3 is separated from the flanges of the upper trapezoidal boss 2.1 of the two connecting blocks, and the lower clamping block 3 is driven to moved upward, so that the concave edge of the trapezoidal groove 3.1 of the lower clamping block 3 is separated from the flanges of the lower trapezoidal boss 2.1 of the two connecting blocks, respectively, and the two connecting plates can be opened.

Embodiment 2 is shown in FIG. 4.

A folding joint includes two connecting plates 2 connected to two sections of rods 1, respectively. Sides of the two connecting plates 2 are hinged to each other, and opposite cooperating surfaces of the two connecting plates 2 are provided with an upper trapezoid boss 2.1 and a lower trapezoid boss 2.1 outside the two connecting plates 2. Movable clamping blocks 3 are arranged at the upper and lower bosses of one connecting plate 2, and a side of the clamping block 3 adjacent to the trapezoidal boss 2.1 is provided with a trapezoidal groove. An upper section of a screw rod 4 has a right-handed thread connected to a right-hand thread of the lower clamping block 3, and a lower section of the screw rod 4 has a left-handed thread connected to a left-hand thread of the lower clamping block 3. An upper section of screw rod 4 is provided with an inner hexagonal hole, and the screw rod 4 is fixedly connected to the connecting plate 2 through a screw rod buckle 6.

According to the folding joint, the screw rod 4 is driven to rotate using an external angle wrench or a socket wrench, such as a hexagonal wrench 9. When the screw rod 4 is rotated rightwards by the hexagonal wrench 9, the right-handed thread of the screw rod 4 drives the upper clamping block 3 to move upward, so that the concave edge of the trapezoidal groove 3.1 of the lower clamping block 3 is in contact with the flanges of the lower trapezoidal boss 2.1 of the two connecting blocks, respectively, which is in a locked state. A spring 5 is provided on the screw rod, and the spring 5 keeps pressing the clamping block to prevent the screw rod 4 from rotating leftwards accidentally and causing the clamping block 3 to move, which plays a safe role. When the screw rod 4 is rotated leftwards by the hexagonal wrench 9, the right-handed thread of the screw rod 4 drives the upper clamping block 3 to move down, so that the concave edge of the trapezoidal groove 3.1 of the upper clamping block 3 is separated from the flanges of the upper trapezoidal boss 2.1 of the two connecting blocks, and the lower clamping block 3 is driven to moved upward, so that the concave edge of the trapezoidal groove 3.1 of the lower clamping block 3 is separated from the flanges of the lower trapezoidal boss 2.1 of the two connecting blocks, respectively, and the two connecting plates can be opened.

1. A folding joint comprising two connecting plates connected to two sections of rods respectively, sides of the two connecting plates being hinged to each other, wherein at least two corresponding edges of opposite cooperating surfaces of the two connecting plates are provided with bosses, each boss of one of the connecting plates is provided with a movable clamping block with a groove on a side thereof, a concave edge of the groove on the side of the clamping block is capable of being in contact with a flange of each boss of the connecting plate simultaneously when the two connecting plates are close together, the clamping block is respectively driven by left and right threads on a screw rod, so that the concave edge of the groove of the clamping block is in contact with or moves away from the flange of the boss of the connecting plate.

2. The folding joint according to claim 1, wherein the boss of the connecting plate is arranged inside the connecting plate.

3. The folding joint according to claim 1, wherein the boss of the connecting plate is arranged outside of the connecting plate.

4. The folding joint according to claim 2, wherein the flange of the boss of the connecting plate is trapezoidal or arc-shaped.

5. The folding joint according to claim 4, wherein the two connecting plates are hinged through a biaxial connecting block and two hinge shafts.

6. The folding joint according to claim 5, wherein the screw rod is driven to rotate by an external angle wrench or a socket wrench.

7. The folding joint according to claim 5, wherein an upper section of the screw rod is fixedly connected to a handle.

8. The folding joint according to claim 6, wherein a spring is provided on the screw rod to press the clamping block.

9. The folding joint according to claim 7, wherein a safety buckle seat and a safety buckle are provided on the rod.
